(19) United States
(12) Patent Application Publication
(10) Pub. No.: US 2023/0243821 A1
(43) Pub. Date: Aug. 3, 2023

(54) BLOCKING METHOD

(71) Applicants: QBD (QS-IP) LIMITED, St Helier (JE); QUOTIENT SUISSE SA, Eysins (CH)

(72) Inventors: Emmet McGauran, Eysins (CH); Yohann Dupont, Eysins (CH)

(21) Appl. No.: 18/004,541

(22) PCT Filed: Jul. 9, 2021
(86) PCT No.: PCT/EP2021/069222

§ 371 Date: Jan. 6, 2023

(30) Foreign Application Priority Data
Jul. 10, 2020 (GB) 2010627.4
Oct. 23, 2020 (GB) 2016865.4

(51) Int. Cl. G01N 33/543; B05D 1/12; B05B 13/02; B05B 17/06

(52) U.S. Cl. CPC G01N 33/54393; B05B 13/0221; B05B 17/06; B05D 1/12

(57) ABSTRACT

A method of applying a blocking composition (116) on a substrate for an assay (20) comprises: providing a substrate for an assay (20), wherein the substrate for an assay comprises a solid substrate provided with a plurality of discrete spots of a biological material on a surface thereof; and spraying the blocking composition (116) onto the substrate (20) as particles or droplets having a diameter being less than the diameter of the printed spots of biological material.

### FIELD

The present disclosure relates to a method and apparatus for applying a blocking composition on a substrate for an assay such as a microarray.

### BACKGROUND

A microarray is a two-dimensional array of a biological material, such as proteins, DNA, antigens or antibodies, deposited and immobilised on a solid and typically flat substrate, typically made of functionalized glass or plastic. The substrate is generally selected to provide adequate binding with the biological material, but also to avoid any modification of the material, such as denaturation in the case of a protein.

Typically, once the microarray of biological material has been deposited on the substrate, a blocking buffer is applied to the microarray surface to prevent unwanted materials binding to the surface of the substrate. The microarray is then reacted with a sample, and detection is carried by a suitable microarray analysis technique, for example dark field microscopy. An example of such microarray-based technology used for blood grouping and donor disease screening is MosaiQ™ by Quotient.

The binding of unwanted materials to the surface of the substrate is often referred to as “non-specific binding”. An effect of non-specific binding is that it causes high backgrounds levels during detection and hence can impact on the ability to detect a positive response (foreground) from background signal. The quality of the desired analyte signal to unwanted background is usually termed “signal-to-noise ratio”. Too high a level of background and/or a low signal-to-noise ratio can lead to aberrant results, including, for example, so-called “false positive” or “false negative” results

In order to mitigate non-specific binding and reduce false negative results, it is common to increase the concentration of the biological material on the substrate so as to increase contrast during subsequent optical detection. Although this may improve sensitivity, this approach may cause a problem of reduced specificity by increasing the occurrence of false positive results. This “false positive results” problem caused by an increased concentration of the printed biological material typically manifests itself as the so-called “Comet Tail” effect. Comet Tails are an effect of reactions between biological material, e.g. an antibody, originally printed on the substrate as part of the microarray but which has migrated outside of its intended specific print area, and subsequently resulting in comet tail phenomenon during the subsequent reaction with a dedicated sample of interest. The comet tail phenomenon has the capability to impact the result of neighbouring print areas and increase the uncertainty of the reaction state of the printed area of interest. Without wishing to be bound by theory, it is believed that such Comet Tails may result from the application process of the blocking buffer on the microarray. An excessively long period of blocking can cause washing-off of the spotted DNA. Microarrays are particularly prone to this problem because the surface of the microarray is typically flat or substantially flat. This phenomenon is called “comet tails” (Eisen, M.B. and Brown, P.O. (1999) DNA Arrays for Analysis of Gene Expression, Methods in Enzymology, 303, 179-205.) Others have suggested that “comet-tails” may be due to the high concentration of biological material (being, e.g. an antibody), used during spotting.

EP2640876A1 (Honkanen et al) and EP2603325A1 (Oliver et al) disclose methods for printing in-well calibration features onto assay substrates. Whilst a blocking material is applied to the target plate, the plate is formed of a plurality of wells and therefore does not suffer from potential “comet tail” effect like a microarray, and this document does not provide any solution to address this problem.

WO2013/037886 (Caussette et al) discloses methods for detecting analytes in a sample. Whilst a blocking material is sprayed onto a nitrocellulose membrane, the membrane does not suffer from potential “comet tail” effect like a microarray, and this document does not provide any solution to address this problem.

It is an object of the present invention to obviate and/or mitigate the limitations and/or disadvantages associated with the prior art and/or with conventional methods and systems.

### SUMMARY

According to a first aspect there is provided a method of applying a blocking composition on a substrate for an assay, the method comprising:
providing a substrate for an assay; and
spraying the blocking composition onto at least a portion of the substrate.

The method may comprise spray coating the blocking composition onto at least a portion of the substrate.

The substrate for an assay may comprise an immobilised substance or composition. The substance or composition may be immobilised to a surface of the substrate. The substance or composition may be immobilised at one or more discrete locations on (a surface of) the substrate. The substance or composition may be immobilised in the form of an array - i.e. at a plurality of discrete locations on (the surface of) the substrate. The substrate may be provided with a one-dimensional, two-dimensional, or three dimensional array of the substance or composition on a surface thereof. Typically, the substrate may be provided with a two-dimensional array of the substance or composition on a surface thereof. Typically also, a surface of the substrate, e.g. a surface on which the substance or composition is immobilised, is flat or planar or is substantially flat or planar. The terms “flat” and “planar” will be understood to refer to the shape of the surface at a macroscopic level, i.e., it be herein understood to mean that the surface does not include any wells or grooves that may be configured to receive a substance or composition. Rather, the substance or composition is immobilised at different and/or discrete locations of the substrate on an otherwise substantially continuous surface. At least a portion of the substrate, e.g. a portion of the surface of the substrate configured to receive the substance or composition, may be free of wells or recesses.

The substance or composition may comprise a biological material.

The term “biological material” will be herein understood as encompassing any biological material, whether naturally or synthetically made.

Typically, the biological material may comprise a peptide, a protein (including recombinant proteins), an amino-acid, a nucleic acid (DNA and/or RNA), an oligonucleotide, a lipid, a carbohydrate, an enzyme, a metabolite, an antibody (including monoclonal and/or polyclonal antibodies and any (antigen binding) fragments thereof), an antigen, cells, red blood cells, plasma, serum or the like.

In an embodiment, the biological material may comprise, consist essentially of or consist of antibodies.

In another embodiment, the biological material may comprise, consist essentially of, or consist of red blood cells.

The substrate may be referred to as a ‘chip’.

There may be provided a single substrate, e.g. chip. In such instance the method may comprise spraying the blocking composition onto the single substrate, e.g. chip.

There may be provided a plurality of substrates, e.g. chips. In such instance the method may comprise spraying the blocking composition onto the plurality of substrates, e.g. chips.

The method may comprise spraying the blocking composition onto at least a portion of the biological material.

The substrate for an assay may comprise a solid substrate provided with a plurality of discrete amounts, e.g. spots, of a biological material (immobilised) on a surface thereof, e.g. on a substantially flat surface thereof.

The substrate for an assay may comprise or may be a microarray.

Typically, the size, e.g. diameter, of the discrete amounts, e.g. spots, of biological material immobilised to the surface of the substrate may be approximately 100 µm -300 pm, e.g. about 150 µm - 250 µm, typically about 210 µm +/-40 µm.

The blocking composition may comprise, may consist of or may consist essentially of a blocking buffer.

It will be appreciated that the choice of blocking composition applied on the substrate, e.g. microarray may depend on the microarray being used, and in particular, on the substrate material, biological material, and/or or the sample being assayed.

The blocking composition, e.g. blocking buffer, may comprise a blocking agent such as Bovine serum albumin (BSA), serum and/or fish gelatin. The blocking composition may further comprise a base buffer such as a Tris-buffered saline (TBS) or a phosphate-buffered saline (PBS).

Typically, the solid substrate may be made of glass, silicon, or a polymer such as nitrocellulose.

The solid substrate may optionally be coated with a coating layer which may be selected so as to improve or alter properties of or interaction with the biological material, including adhesion, immobilisation, stabilisation, etc. The coating layer may comprise, may consist essentially of or may consist of a metal such as aluminium or gold, a polymer such as hydrophilic polymers or hydrophilic polymer, e.g. polyacrylamide, or the like.

The method may comprise spraying, e.g. spray coating, the blocking composition onto the microarray using a spray coating device.

The spray coating device may be capable of coating the blocking composition onto the substrate, e.g. microarray, to provide the required blocking function. Without wishing to be bound by theory, it is believed that spray coating the blocking composition onto the microarray may avoid, reduce or minimise undesired migration of biological material not bound to the substrate from its original microarray location or spot. This may help reduce, minimise or avoid any “comet tail” effect. Thus, advantageously, the present method may help maintain or preserve the integrity of the biological material provided on the substrate. The present method may also improve the uniformity of the blocking composition applied on the substrate. The present method may reduce, avoid and/or prevent the occurrence of aberrant results, including, for example, so-called “false positive” or “false negative” results. The present method may improve the sensitivity and/or specificity during subsequent analysis, e.g. detection and/or measurement.

The spray coating device may comprise a pressure-driven, e.g. an air pressure-driven spray coating device. The spray coating device may comprise a spray valve. The spray coating device may comprise an atomizer comprising a nozzle controller. The nozzle controller may be configured to control and/or regulate liquid pressure between 0 and 2 bars and/or to control and/or regulate air pressure between 0 and 7 bars. The nozzle controller may be configured to control and/or regulate air pressure at about 1 bar (100 kPa +/- 20 kPa). The spray coating device, e.g. nozzle controller, may be configured to provide a spray rate of about 1 ml/min to about 10 ml/min.

The spray coating device may comprise an ultrasonic spray coating device, e.g. an ultrasonic atomiser. The spray coating device may comprise an ultrasonic atomiser. The ultrasonic atomizer may operate at a frequency of about 20 kHz to 40 kHz. The ultrasonic atomizer may comprise a piezoelectric converter, which may be made of PZT (Zirconate Titanate Crystals) and may be configured to process liquid volume from about 250 µl to about 10 ml.Typically, the density of the blocking composition may be in the range of about 0.98 g/mL to 1.10 g/mL, e.g. between about 0.99 g/mL & 1.08 g/mL (at 20° C.), e.g. between about 0.9982 g/mL & 1.06 g/mL (at 20° C.). The ultrasonic atomiser may be configured to provide a spray rate of about 3 ml/min to about 6 ml/min.

The method may comprise spray coating the blocking composition as particles, e.g., droplets having a size, e.g. diameter, that is less than the size, e.g. diameter, of the printed areas, e.g. spots, of biological material on the microarray. Advantageously, this may help maintain or preserve the integrity of the biological material provided on the substrate.

The method may comprise spray coating the blocking composition as particles, e.g. droplets, having a size, e.g. diameter, between about 1 µm and about 200 µm, for example measured as Mass Median Diameter (MMD).

When the spray coating device is a pressure-driven spray coating device, the method may comprise spray coating the blocking composition as droplets having a size, e.g. diameter, of about 50-150 micrometers, typically about 100 micrometers. It will be appreciated that the size of the droplets may vary depending on certain parameters set by a user, such as pressure and/or volume/rate settings.

When the spray coating device is an ultrasonic spray coating device, e.g. an ultrasonic atomiser, the method may comprise spray coating the blocking composition as droplets having a size, e.g. diameter, of about 20-100 micrometers, typically about 50 micrometers. It will be appreciated that the size of the droplets may vary depending on certain parameters set by a user, such as frequency and/or volume/rate settings.

The method may comprise spray coating the blocking composition as an aerosol.

The method may comprise spraying, e.g. spray coating, the blocking composition at an angle relative to the substrate between about 0° and 180°, typically between about 10° and 170°.

The method may comprise spraying, e.g. spray coating the blocking composition from a projection height of about 0 mm to about 1000 mm, typically from about 1 mm to about 750 mm.

When the spray coating device is a pressure-driven spray coating device, the method may comprise spray coating the blocking composition from a projection height of about 100 mm to about 1000 mm, typically from about 500 mm to about 750 mm. Typically, method may comprise spray coating the blocking composition from a projection height of about 60 cm +/- 15 cm.

When the spray coating device is an ultrasonic spray coating device, e.g. an ultrasonic atomiser, the method may comprise spray coating the blocking composition from a projection height of about 0 mm to about 750 mm, typically from about 1 mm to about 500 mm. Typically, when the spray coating device is an ultrasonic atomiser, the method may comprise spray coating the blocking composition from a projection height of about 5 cm to about 15 cm.

The method may comprise spraying, e.g. spray coating the blocking composition substantially uniformly over the surface of the substrate, e.g. over at least the surface area of the substrate on which the biological material has been deposited.

The spray coating device may be either pressure-driven or ultrasonic, and the method may comprise spray coating the blocking composition from a projection height selected so as to provide uniform coverage of the substrate.

Thus, advantageously, the present invention provides improved uniformity of coverage of the surface of the substrate by the blocking composition.

Without wishing to be bound by theory, it is believed that the use in the spray coating method of smaller droplet size and/or of lower velocity, may improve coating uniformity and consistency with the blocking composition whilst also reducing the risk of damage to the spots of the biological material immobilised on the surface of the substrate.

Advantageously, the spray coating device may comprise an ultrasonic atomiser. By such provision, the method may comprise spray coating the blocking composition at a reduced velocity, with droplets having a small size (typically about 20-100 micrometers, e.g. about 50 micrometers), and from a low projection height (typically about 5 cm to about 15 cm). This may contrast with conventional approaches that use spraying at high velocity from higher projection height and with larger particles, which may lead to “bouncing off” of the droplets on the substrate surface and/or to disruption and/or damage of the biological material spots immobilised on the surface. Thus, the present method may provide improved integrity, uniformity, and consistency, of coating of the substrate, e.g. microarray, by the blocking composition.

The method may comprise spraying the blocking composition discretely over one or more spots, e.g. over each spot, of the biological material which has been deposited on the substrate.

The method may comprise spraying, e.g. spray coating, the blocking composition over an area substantially limited to the surface area of the substrate on which the biological material has been deposited (herein termed “printed area”). This may help to preserve surface tension over the spray coated area.

The method may comprise spraying, e.g. spray coating, the blocking composition over an area including the surface area of the substrate on which the biological material has been deposited, but excluding an area of the microarray devoid of biological material (which may be termed “waste area”). The method may comprise spraying, e.g. spray coating the blocking composition over an area including the surface area of the substrate on which the biological material has been deposited, but excluding a peripheral area of the microarray substrate.

The method may comprise spraying, e.g. spray coating, a first blocking composition onto a first portion of the biological material provided or immobilised on the substrate. The method may comprise, e.g. subsequently, spraying a second blocking composition (which may be the same as or different from the first blocking composition) onto a second portion of the biological material provided or immobilised on the substrate.

Typically, the area of the microarray substrate not coated by the blocking composition may extend outwards from about 5 mm, e.g. from about 10 mm, e.g. from about 50 mm, of the printed area.

Typically, the area of the microarray substrate treated, e.g. coated, by the blocking composition may have a size of about 100 mm × 100 mm to about 150 mm × 150 mm, typically about 128 mm × 128 mm.

The method may comprise spraying, e.g. spray coating a volume of the blocking composition between about 0.5 mL and about 20 mL, e.g. between about 1 mL and about 10 mL, typically between about 1.0 mL and about 8.6 mL, per substrate.

The method may further comprise:
applying a sample on the substrate, and
analysing the substrate.

Typically, the analysis step comprise detecting and/or measuring the sample on the substrate.

Advantageously, the blocking method of the present invention may help improve specificity and/or sensitivity during the subsequent analysis step, e.g. during detection and/or measurement.

The method may reduce, avoid and/or prevent the occurrence of aberrant results, including, for example, so-called “false positive” or “false negative” results. The method may reduce, avoid and/or prevent the occurrence of a “comet tail” effect.

According to a second aspect there is provided a method of applying a blocking composition on a substrate for an assay, the method comprising:
providing a substrate for an assay, wherein the substrate for an assay comprises a solid substrate provided with a plurality of discrete spots of a biological material on a surface thereof, and wherein the surface of the substrate is substantially planar; and
spraying a blocking composition onto at least a portion of the substrate.

Advantageously, the method may allow preserving the integrity of a biological material immobilised on the substrate.

The method may comprise spraying the blocking composition onto at least a portion of the biological material.

The method may further comprise:
applying a sample on the substrate, and
analysing the substrate.

Typically, the analysis step comprise detecting and/or measuring the sample on the substrate.

Advantageously, the blocking method of the present invention may help improve specificity and/or sensitivity during the subsequent analysis step, e.g. during detection and/or measurement.

The method may reduce, avoid and/or prevent the occurrence of aberrant results, including, for example, so-called “false positive” or “false negative” results. The method may reduce, avoid and/or prevent the occurrence of a “comet tail” effect.

Thus, in a third aspect, there is provided a method of reducing, avoiding and/or preventing the occurrence of aberrant results, the method comprising:
providing a substrate for an assay, wherein the substrate for an assay comprises a solid substrate provided with a plurality of discrete spots of a biological material on a surface thereof, and wherein the surface of the substrate is substantially planar; and
spraying a blocking composition onto at least a portion of the substrate.

The method may further comprise:
applying a sample on the substrate, and
analysing the substrate.

The features described in respect of the first aspect may equally apply to the method according to the second aspect or the third aspect, and are not repeated here merely for reasons of brevity.

According to a fourth aspect there is provided an apparatus for applying a blocking composition on a substrate for an assay, the apparatus comprising:
a substrate support for receiving the substrate for an assay; and
a spraying device configured to spray the blocking composition on the substrate.

Typically, the substrate, e.g. microarray, may be provided on a substrate holder, e.g. microarray holder.

The substrate support may have a receiving element configured to support the substrate, substrate holder, microarray or microarray holder.

The substrate support may have a holding element configured to hold the substrate, substrate holder, microarray or microarray holder in position, in use. The holding element may comprise or may be provided in the form of a pair of guides attached to the receiving element.

The holding element may be sized so as to receive a substrate, substrate holder, microarray or microarray holder of a conventional size. The holding element may be adjustable so as to receive substrates, substrate holders, microarrays and/or microarray holders of different sizes.

The apparatus, e.g. substrate support, may comprise a masking element configured to be positioned over the substrate, substrate holder, microarray holder or microarray. The masking element may be configured to cover at least part of the substrate, substrate holder, microarray holder or microarray. The masking element may be configured to cover the microarray holder and part of the microarray.

The masking element may be configured to cover a peripheral region of the substrate or microarray which is devoid of any biological material on its surface.

The masking element may be configured to leave unmasked a first portion of biological material provided or immobilised on the substrate, and/or to cover a second portion of the biological material provided or immobilised on the substrate. This may allow a first blocking composition to be sprayed onto a/the first portion the substrate which is not covered by the masking element. Subsequently, a second blocking composition (which may be the same as or different from the first blocking composition) may be sprayed onto the second portion.

The microarray holder and/or part of the microarray may be sandwiched between the receiving element and the masking element. The substrate and/or part of the substrate holder may be sandwiched between the receiving element and the masking element.

The masking element may be detachably attachable, e.g. by pins, lugs, clips, screws or the like, to the receiving element.

The masking element may have an opening near a central region thereof. The opening may be sized so as to substantially match the area of the microarray to be coated with a blocking buffer. By such provision, the masking layer may protect the areas of the microarray which should remain free of blocking buffer.

Typically, the opening may have a size of about 100 mm × 100 mm to about 150 mm × 150 mm, e.g. about 128 mm × 128 mm.

The apparatus may comprise an enclosure, typically a rigid enclosure, configured to at least partially surround or contain the spray coating device. The enclosure may be configured to maintain, secure, control and/or adjust the position of the spray coating device. The enclosure may be configured to limit, reduce and/or minimise turbulence within the enclosure and/or between the spraying device and the substrate, which may help improve quality of the spraying method, including for example control uniformity and/or repeatability of the spray coating process.

The apparatus enclosure may provide a substrate support for receiving and/or securing the microarray holder and/or microarray.

The enclosure may include a frame, which may typically be of a cuboidal shape. The frame may include or may be connected to one or more feet.

The enclosure may be provided with one or more sides or panels configured to at least partially confine any sprayed material to an inside portion of the enclosure. Conveniently, at least one side or panel, e.g. the sides or panels, may be transparent or translucent so as to allow a user to view and/or monitor an inside portion of the enclosure. The one or more sides or panels may be made of glass or plastic.

The enclosure may comprise a rear panel and a side panel on each side of the rear panel. The enclosure may a door on a front side thereof. The door may be made of a transparent or translucent material, e.g. glass or plastic, in order to allow a user to view and/or monitor inside the enclosure. The door may allow access to an interior portion of the enclosure. The door may be hinged to the enclosure, e.g. frame thereof.

Typically, the enclosure, e.g. frame, may have a height of about 50-150 cm, e.g. 50-100 cm, typically about 75 cm.

Typically, the enclosure, e.g. frame, may have a width of about 20-100 cm, e.g. 25-75 cm, typically about 50 cm.

Typically, the enclosure, e.g. frame, may have a depth of about 20-100 cm, typically about 50 cm.

The enclosure may include a support frame, typically near a rear portion thereof, configured to connect and secure a/the spraying device. Advantageously, the support frame may be configured to connect and secure a first mounting plate.

The first mounting plate may be configured to support a first spraying device.

The support frame may include or may be formed or struts or rails, which may be attached to the frame or may form an integral part of the frame. Advantageously, the support frame, e.g. rails or struts thereof, may be movable relative to the frame, e.g. in two dimensions and/or in a vertical plane, so as to allow adjustment of height and width thereof.

Conveniently, there may be provided a first connecting element, e.g. first articulated arm, which may be attached to the first mounting plate, e.g. to apertures therein. The first connecting element, e.g. articulated arm, may be configured to attach to the first spraying device. The first connecting element may be configured to allow adjustment of the position, height, and/or spraying angle of the first spraying device .

The first mounting plate may be disposed substantially vertically and/or substantially parallel to the rear panel.

The first spraying device may comprise or may be an ultrasonic spray coating device, e.g. an ultrasonic atomiser.

The apparatus may comprise a second mounting plate configured to support a second spraying device. The second mounting plate may configured to act as an upper panel of the enclosure, and may be disposed substantially horizontally, in use. The second mounting plate may be attached to an upper portion of the frame.

Conveniently, there may be provided a second connecting element, e.g. second articulated arm, which may be attached to the second mounting plate, e.g. to apertures therein. The second connecting element, e.g. second articulated arm, may be configured to attach to the second spraying device.

The second connecting element may be configured to allow adjustment of the position, height, and/or spraying angle of the second spraying device.

The second spraying device may comprise or may be an air pressure-driven spray coating device, e.g. a spray coating device having a spray valve.

The apparatus may comprise the first spraying device, the second spraying device, or both. In other words, the term “second” as used herein does not necessarily imply the presence of the features referred to as “first” such as first spraying device, first mounting plate, and/or first connecting element.

For example, if no second spraying device is used and/or if only the first spraying device is used, there may not be provided a second mounting plate and/or the apparatus may comprise an upper panel connected to an upper portion of the frame. The upper panel may be made of a transparent or translucent material such as glass or plastic.

The apparatus may comprise a movable platform configured to support the substrate support, e.g. the receiving element thereof.

The movable platform may comprise one or more moving means, e.g. wheels or bearings, to allow the movable platform to be moved in a first direction.

The movable platform may comprise one or more moving means, e.g. wheels or bearings, to allow the movable platform to be moved in a second direction.

The movable platform may comprise one or more wheels to allow the movable platform to be moved in a first direction and bearings to allow the movable platform to be moved in a second direction. The second direction may be transverse, e.g. perpendicular, to the first direction.

The movable platform may include control means to control or operate movement of the moveable platform. By such provision, a user may be able to control the application of a blocking buffer by the spraying device, for example by controlling the duration of application and the precise area of the microarray being treated.

Movement of the moveable platform may be operated or controlled manually. The control means may comprise a handle to allow a user to move the platform.

Movement of the moveable platform may be operated or controlled electronically and/or remotely. The control means may comprise a user interface which may be in communication, e.g. in wired or wireless communication, with the moveable platform. Conveniently, the user interface may be located outside the enclosure so as to confine the blocking buffer within the enclosure, in use.

The apparatus may include a receptacle, e.g. a tray, configured to collect excess blocking buffer dispensed by the spraying device.

The apparatus may further comprise a permeable sheet or shelf configured to support the movable platform and to allow drainage of excess blocking buffer through the shelf. The provision of a permeable sheet or shelf may provide the movable platform with a suitable support surface for moving, e.g. rolling, whilst permitting excess liquid therethrough, e.g. into the receptacle, e.g. tray.

For the avoidance of doubt, any feature described in respect of any aspect of the invention may be applied to any other aspect of the invention, in any appropriate combination. For example, method features may be applied to apparatus features and vice versa.

### BRIEF DESCRIPTION OF THE DRAWINGS

The present invention will now be further described in detail and with reference to the figures in which:

FIGS. 1 and 2 shows a conventional method of applying a blocking buffer on a microarray according to the prior art;

FIG. 3 shows an image of a reacted microarray illustrating a “Comet Tail” effect;

FIG. 4 illustrates a method of applying a blocking buffer on a microarray according to a first embodiment;

FIGS. 5a, 5b, 6a, 6b, 7a, 7b, 8a and 8b illustrate a comparison of the assay images obtained using a conventional blocking method and the method of FIG. 4, for four different samples;

FIG. 9 is a graph comparing the occurrence of comet tails obtained using a conventional blocking method and the method of FIG. 4, for different samples;

FIG. 10 shows a front view of an apparatus according to an embodiment;

FIG. 11 shows a part-exploded perspective front view of the enclosure of the apparatus of FIG. 10;

FIG. 12 shows a rear perspective view of the enclosure of the apparatus of FIG. 10;

FIG. 13 shows a front view of the apparatus of FIG. 10, showing an ultrasonic atomiser and a substrate support;

FIGS. 14 to 16 show illustrations of an embodiment of the substrate support of FIG. 13;

FIG. 17 shows a schematic view of another embodiment of a spraying apparatus;

FIG. 18 shows a schematic view of another embodiment of a spraying apparatus.

### DETAILED DESCRIPTION OF THE DRAWINGS

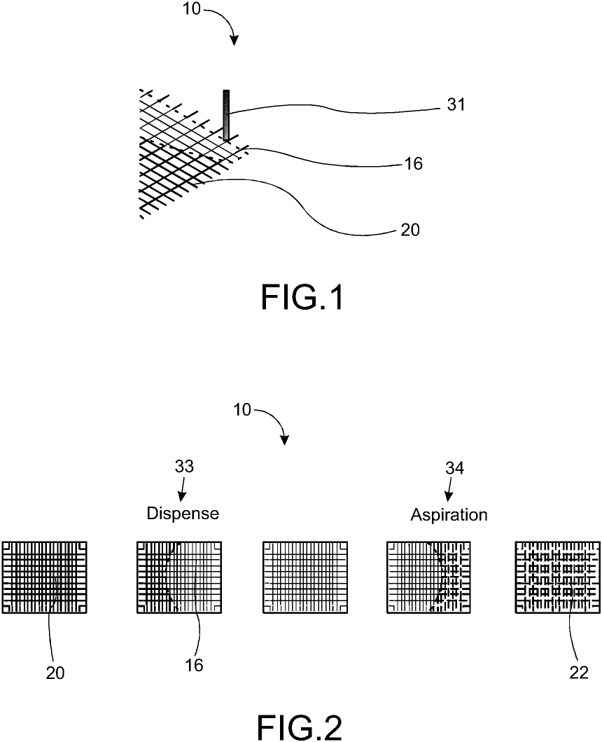

FIGS. 1 and 2 show a conventional (wet) method (10) for applying a blocking buffer to a microarray (20). As shown in FIG. 1, the method comprises a dispensing tip 31 which (as shown in FIG. 2), dispenses a blocking buffer 16 in a dispensing phase 33. The blocking buffer 16 spreads over the surface of the microarray 20 and excess blocking buffer is aspirated during an aspiration phase 34 to yield a blocked microarray 22.

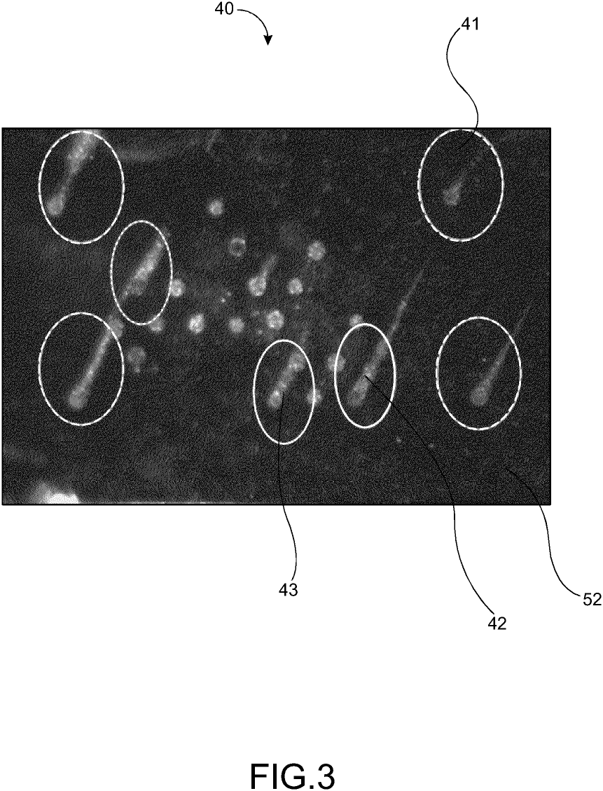

FIG. 3 shows an image of a microarray 40 used to assay reactions between an antibody printed on the microarray and red cells from a sample. FIG. 3 illustrates the “Comet Tail” effect, where it can be seen that the areas showing positive reaction exceed the original printed spot area. In this example, three cell lines were impacted, namely Positive Controls (41), Cell Line “A” (42), and Cell Line “B” (43).

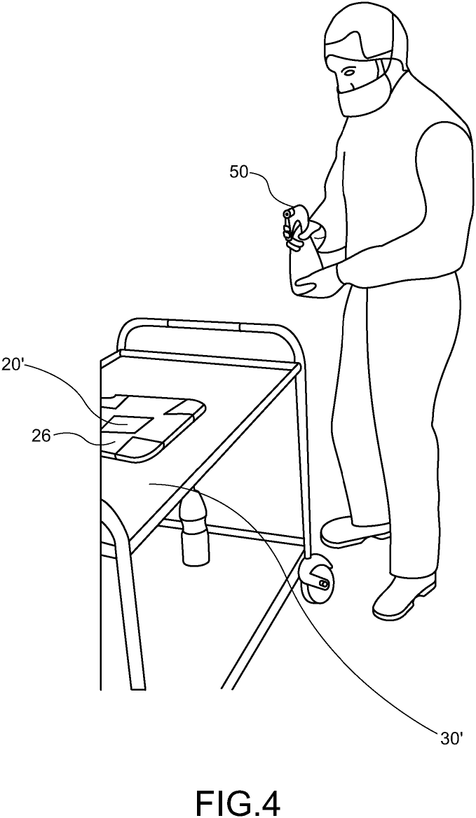

FIG. 4 shows an embodiment of a method according to a first embodiment. In this method, a microarray 20′ was paced on a support surface 30′. The peripheral surface of the microarray 20′ (the “waste area” not printed with any biological material) was protected by a masking element 26. The microarray way was printed with cell lines previously identified as having propensity to cause Comet Tail effect, and printed following an Immunohematology (IH) layout.

The exposed (printed) area of microarray 20 was then sprayed with a blocking buffer (as specified in Table 1 below) using a manual spray device 50, with the nozzle of the device 50 being aligned at approximately 0° (i.e. substantially parallel to the microarray 20), in order to minimise the pressure exerted on the printed spots during the spraying process, and thus reduce the occurrence of “blast spots”.

TABLE 1

Blocking buffer composition (in water)
Components
Bovine Serum Albumin (BSA)
Sodium chloride
Potassium dihydrogen orthophosphate (anhydrous)
Disodium hydrogen orthophosphate (anhydrous)

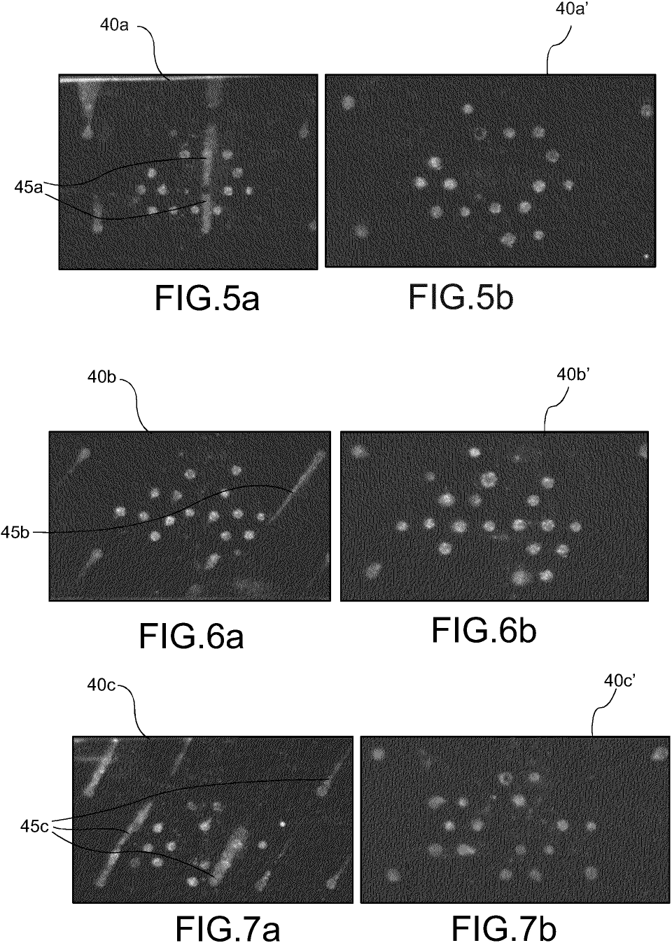

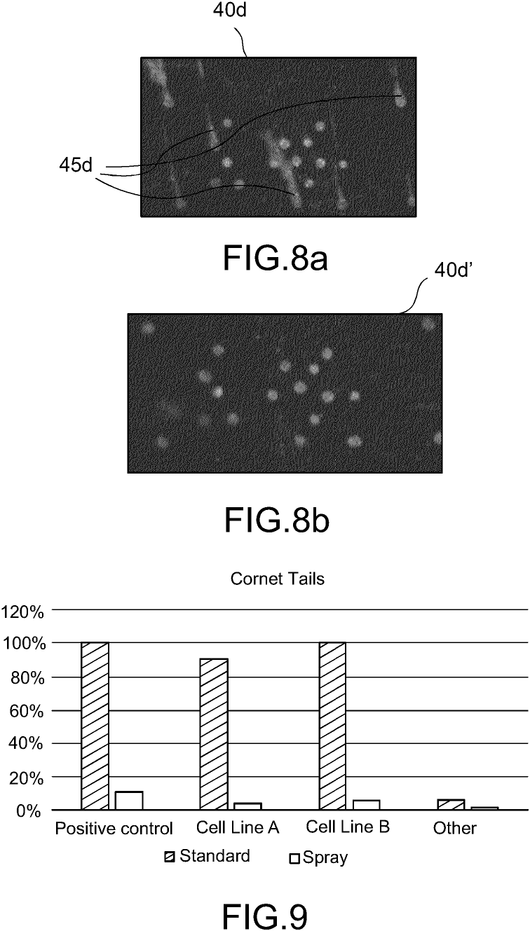

FIGS. 5a, 5b, 6a, 6b, 7a, 7b, 8a and 8b illustrate a comparison of the images obtained using a conventional blocking method and by the method of FIG. 4, for four different samples.

It can be observed that, for each sample, the conventional blocking method led to a number of comet tails 45a,45b,45c,45d, whilst applying the blocking buffer with the spray coating method described in connection with FIG. 4 eliminated the “comet tail” effect entirely, thus improving specificity of the results.

FIG. 9 is a graph comparing the occurrence of Comet Tails obtained using a conventional blocking method and the method of FIG. 4, for different samples.

It can be seen that comet tails occurred in the vast majority of samples prepared using a conventional “spreading” blocking method, whereas comet tails rarely occurred in microarrays blocked using the spray coating method of FIG. 4. Without wishing to be bound by theory, it is believed that the occurrence of comet tails in the samples blocked using the spray coating method of FIG. 4 may be attributed to a non-uniform coverage of the spray, particularly around the peripheral areas of the substrate.

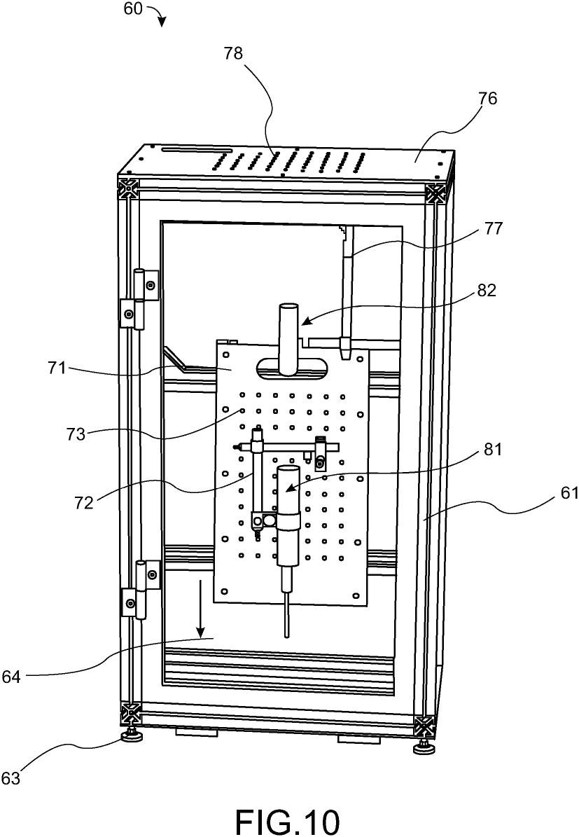

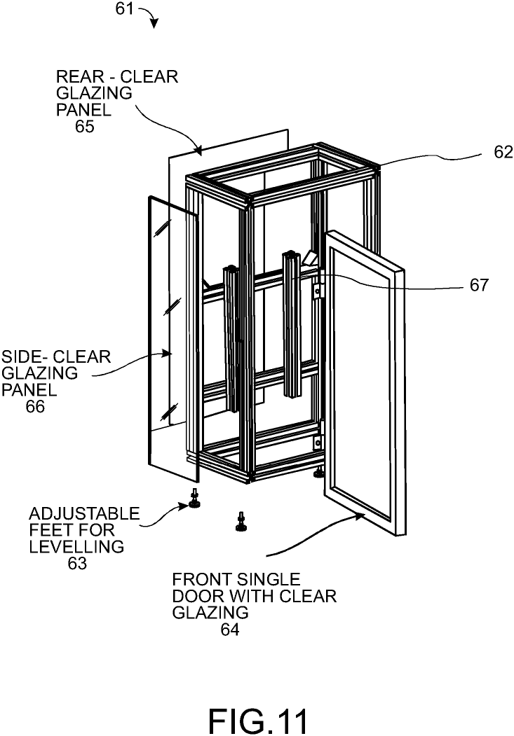

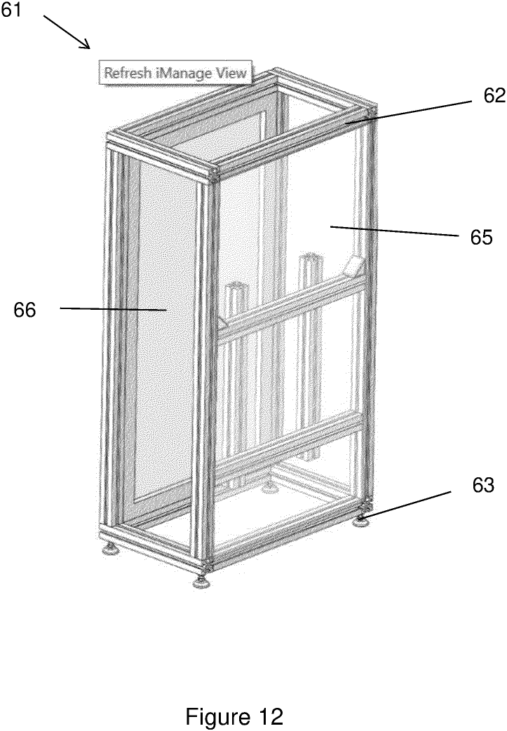

FIG. 10 shows apparatus 60 for spraying a blocking buffer on a microarray, according to an embodiment. The apparatus has an enclosure 61 which in this embodiment is made of metal, but could in other embodiments be made of any other suitable material. As best shown in FIGS. 11 and 12, the enclosure includes a cuboid frame 62 which provides support and structural integrity. The frame 62 is supported on feet 63 for stability. Conveniently, the apparatus 60 is provided with transparent or translucent panels including a rear panel 65 and two side panels 66 so as to allow a user to view and/or monitor the inside of the enclosure 61, in use, whilst confining any sprayed material to the inside of the enclosure 61. There is also provided a front door 64 which is also made of a transparent or translucent material such as glass or plastic in order to allow a user to view and/or monitor inside the enclosure 61.

The enclosure 61 has a support frame 67 which includes struts or rails attached to the frame 62 (but may in other embodiments form an integral part of the frame 62), and which is configured to connect and secure a first mounting plate 71. Advantageously, the rails of the support frame 67 are movable relative to the frame 62, e.g. in two dimensions and/or in a vertical plane, so as to allow adjustment of height and width thereof. The first mounting plate 71 is configured to support a first spraying device 81 via connecting arms 72 which are attached to apertures 73 in the first mounting plate 71. In this embodiment, the first mounting plate 71 is disposed substantially vertically, and the first spraying device 81 is an ultrasonic spray coating device, e.g. an ultrasonic atomiser.

The apparatus 60 also has a second mounting plate 76 which in this embodiment also acts as an upper panel and is attached to an upper portion of the frame 61. The second mounting plate 76 is configured to support a second spraying device 82 via connecting arms 77 which are attached to apertures 78 in the second mounting plate 76. In this embodiment, the second mounting plate 76 is disposed substantially horizontally, and the second spraying device 82 is an air pressure-driven spray coating device, e.g. spray coating device having a spray valve.

The connecting arms 72,77 allow adjustment of the position, height, and spraying angle of a respective spraying device 81,82.

In FIG. 10, both the first spraying device 81 and the second spraying device 82 are shown for ease of understanding. However, it will be appreciated that, in use, a user may choose to select and connect only one type of spraying device, i.e. either first spraying device 81 or the second spraying device 82, depending on the most appropriate type of device required for a specific application.

Typically, the enclosure 61, e.g. frame 62, has a height of about 75 cm, a width of about 50 cm, and a depth of about 50 cm which keeps the overall size of the apparatus 60 relatively compact, whilst permitting a suitable level of adjustment of the spray devices 81,82 in X, Y, and/or Z directions.

Typically the apparatus 60 allows the nozzle of the spraying devices 81,82, to be positioned at a distance of about 0 mm to about 750 mm, from the substrate.

When the spray coating device is a pressure-driven spray coating device 82, the apparatus 60 allows the nozzle of the spraying device 82 to be positioned at a distance of about 500 mm to about 750 mm form the substrate.

When the spray coating device is an ultrasonic atomiser 81, the apparatus 60 allows the nozzle of the spraying device 81 to be positioned at a distance of about 0 mm to about 500 mm from the substrate.

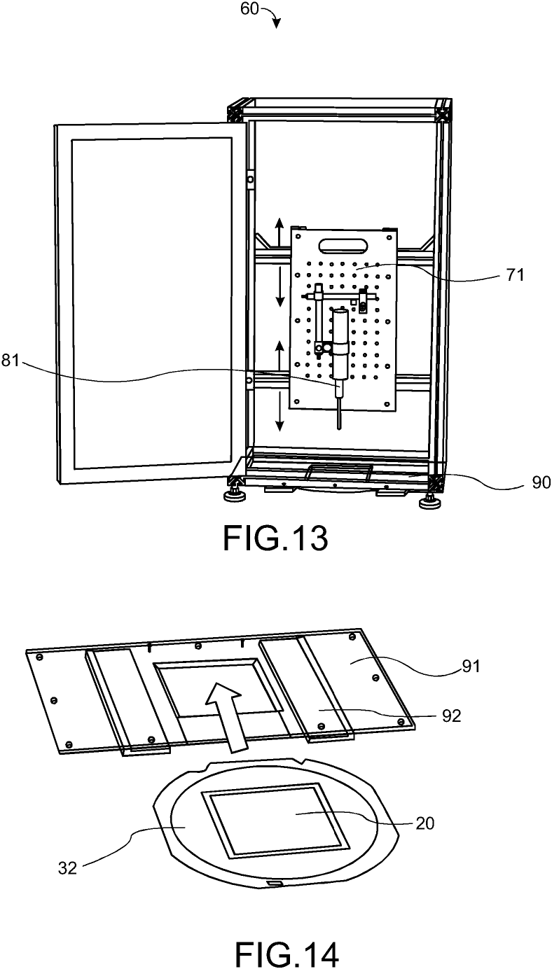

FIG. 13 shows a front view of the apparatus 60 of FIG. 10, showing only the ultrasonic atomiser 81 for clarity, and a substrate support 90 configured to receive a substrate which in this embodiment is a microarray 20 provided on microarray holder 32. As can be seen, the microarray 20 is substantially flat and does not include any wells or recesses.

As shown in FIGS. 14-16, the substrate support 90 has a receiving element 91 configured to support the microarray holder 32. In this embodiment, the receiving element 91 is provided on a lower portion of the frame 62.

Advantageously, the substrate support 90 has a holding element 92 configured to hold the microarray in position, in use. In this embodiment, the holding element 92 is in the form of a pair of guides attached to the receiving element 91. The holding element 92 may be sized so as to receive a microarray slide of a conventional size. In other embodiments, the holding element 92 may be adjustable so as to receive microarrays of different sizes.

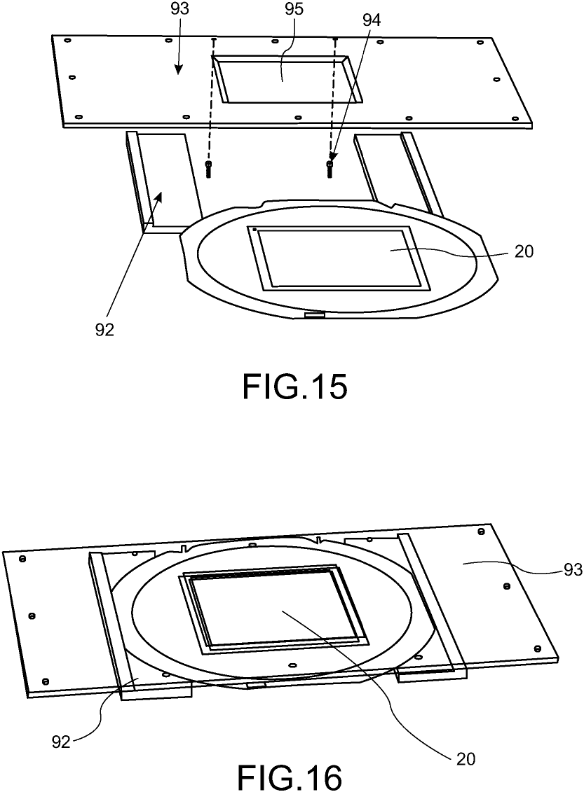

As shown in FIG. 15, there is provided a masking layer 93 configured to be positioned over the microarray holder 32 and to cover part thereof, and in particular to cover a peripheral region of the microarray 20 which is devoid of any biological material on its surface. Thus, in use, the microarray slide may be sandwiched between the receiving element 91 and the masking layer 93.

The masking layer has an opening 95 near a central region thereof. The opening 95 is sized so as to match the area of the microarray 20 to be coated with a blocking buffer. By such provision, the masking layer 93 protects the areas of the microarray slide 20 which should remain free of blocking buffer. The opening typically has a size of about 128 mm × 128 mm (+/- 1 mm).

The location of the masking layer 93 may be secured, in use, by pins 94 which engage an upper surface of the receiving element 91 and engage a lower surface of the masking layer 93.

FIG. 16 shows a view of the assembled substrate support 90 in which the masking layer 93 is depicted in a see-through appearance for ease of visualisation.

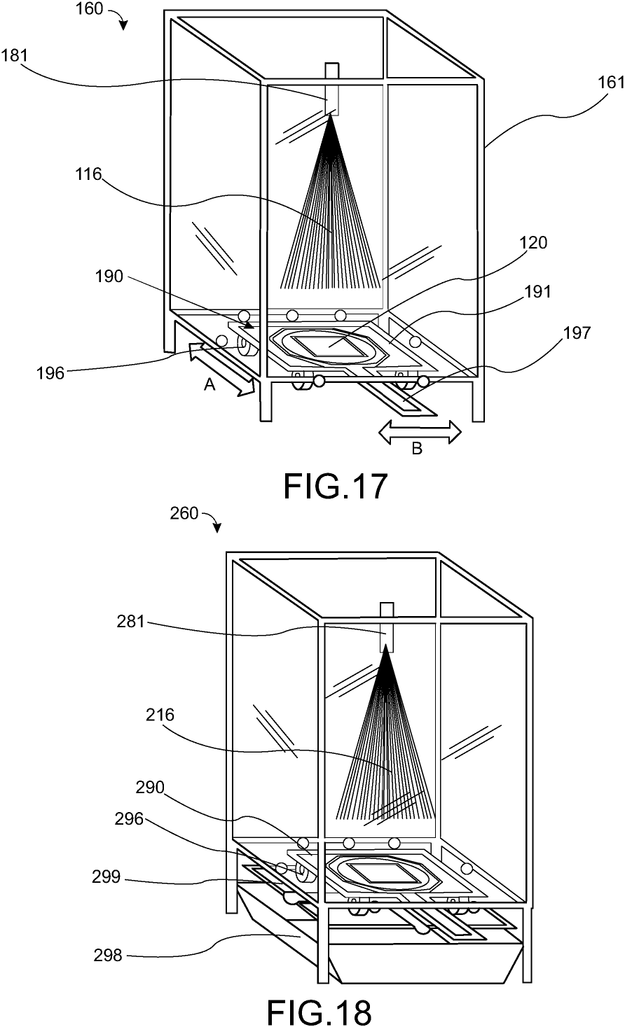

FIG. 17 shows a schematic view of another embodiment of a spraying apparatus 160. The apparatus 160 of FIG. 17 is generally similar to the apparatus 60 of FIG. 10, like parts denoted by like numerals, incremented by ‘100’. In this embodiment, the apparatus 160 has a substrate support 190 which is provided with or connected to wheels 196 to allow the substrate support 190 to be moved in a first direction represented by arrow A. The substrate support 190 is also provided with or connected with bearings (not shown) to allow the substrate support 190 to be moved in a second direction represented by arrow B, which is transverse to the first direction. In this embodiment, the substrate support 190 is connected to a handle 197 that allows a user to control movement of the substrate support 190, e.g. in direction A and/or B.

FIG. 18 shows a schematic view of another embodiment of a spraying apparatus 260. The apparatus 260 of FIG. 18 is generally similar to the apparatus 160 of FIG. 17, like parts denoted by like numerals, incremented by ‘100’. In this embodiment, the apparatus 260 includes a tray 298 below the substrate support 290, configured to collect excess blocking buffer dispensed by the spraying device 281. The apparatus 260 further comprises a permeable sheet 299 configured to support the substrate support 290 and to allow drainage of excess blocking buffer 216 through the sheet 299. The permeable sheet 299 also provides the movable substrate support 290 with a suitable support surface for the wheels 296 allowing movement of the substrate support 290 whilst permitting excess liquid therethrough, e.g. into the tray 298.

It will be appreciated by persons skilled in the art that numerous variations and/or modifications may be made to the invention as described herein without departing from the scope of the present invention. The present embodiments are therefore to be considered for illustrative purposes and are not restrictive, and are not limited to the extent of that described in the embodiment.

1. A method of applying a blocking composition on a substrate for an assay, the method comprising:
providing a substrate for an assay, wherein the substrate for an assay comprises a solid substrate provided with a plurality of discrete spots of a biological material on a surface thereof; and
spraying the blocking composition onto the substrate as particles or droplets having a diameter being less than the diameter of the printed spots of biological material.

2. A method according to claim 1, wherein the method comprises spray coating the blocking composition onto the substrate.

3. A method according to claim 1, wherein the substrate is provided with an array of the biological material on a surface thereof.

4. A method according to claim 1, wherein the substrate for an assay comprises a microarray.

5. A method according to claim 1, wherein the size of the spots of biological material on the surface of the substrate is about 100 µm - 300 µm.

6. A method according to claim 1, wherein the blocking composition comprises, consists essentially of or consists of a blocking buffer.

7. A method according to claim 1, wherein the biological material comprises a peptide, a protein, an amino-acid, a nucleic acid, oligonucleotide, DNA, RNA, a lipid, a carbohydrate, an enzyme, a metabolite, an antibody, an antigen, a cell, red blood cells, plasma, or serum.

8. A method according to claim 1, comprising spraying the blocking composition onto the substrate using an air pressure-driven spray coating device.

9. (canceled)

10. A method according to claim 1, comprising spraying the blocking composition onto the substrate using an ultrasonic atomiser.

11. (canceled)

12. A method of applying a blocking composition on a substrate for an assay, the method comprising:
providing a substrate for an assay, wherein the substrate for an assay comprises a solid substrate provided with a plurality of discrete spots of a biological material on a surface thereof, and wherein the surface of the substrate is substantially planar; and
spraying the blocking composition onto the substrate.

13. A method according to claim 1, comprising at least one of: preserving the integrity of a/the biological material immobilised thereto; and/or improving specificity and/or sensitivity during subsequent analysis.

14. (canceled)

15. A method according to claim 1, wherein the method reduces, avoids and/or prevents the occurrence of aberrant results,
optionally wherein the method reduces, avoids and/or prevents the occurrence of false positive and/or false negative results.

16. A method according to claim 1, further comprising:
applying a sample on the blocked substrate, and
analysing the substrate.

17. An apparatus for applying a blocking composition on a substrate for an assay, the apparatus comprising:
a substrate support for receiving the substrate for an assay; and
a spraying device configured to spray the blocking composition on the substrate, wherein the spraying device is configured to spray the blocking composition as particles or droplets having a diameter being less than the diameter of printed spots of biological material being provided on the substrate.

18. An apparatus according to claim 17, wherein the substrate is provided on a substrate holder, and wherein the substrate support has a receiving element configured to support the substrate holder.

19. An apparatus according to claim 17, wherein the substrate support comprises a masking element configured to be positioned over the substrate or substrate holder, wherein the masking element is configured to cover a peripheral region of the substrate which is devoid of any biological material on its surface, wherein the masking element has an opening near a central region thereof.

20. (canceled)

21. An apparatus according to claim 17, wherein the apparatus comprises an enclosure configured to at least partially surround or contain the spray coating device, wherein the enclosure includes a support frame configured to connect and secure the spraying device, wherein the support frame is configured to connect and secure a first mounting plate, wherein the first mounting plate is configured to support a first spraying device.

22-23. (canceled)

24. An apparatus according to claim 17, wherein the apparatus comprises a first connecting element configured to allow adjustment of the position of the spraying device or first spraying device.

25. An apparatus according to claim 17, wherein the spraying device or first spraying device comprises an ultrasonic atomiser.

26. An apparatus according to claim 17, wherein the apparatus comprises a movable platform configured to receive the substrate support, wherein the movable platform comprises one or more moving means to allow the movable platform to be moved in a first direction, and one or more moving means to allow the movable platform to be moved in a second direction transverse to the first direction.
